(19) United States
(12) Patent Application Publication
(10) Pub. No.: US 2019/0025183 A1
(43) Pub. Date: Jan. 24, 2019

(54) GENE ANALYSIS METHOD

(71) Applicant: FUJIFILM Corporation, Tokyo (JP)

(72) Inventor: Yasuhisa KANEKO, Ashigarakami-gun (JP)

(73) Assignee: FUJIFILM Corporation, Tokyo (JP)

(21) Appl. No.: 16/139,768

(22) Filed: Sep. 24, 2018

Related U.S. Application Data
(63) Continuation of application No. PCT/JP2017/005634, filed on Feb. 16, 2017.

(30) Foreign Application Priority Data
Mar. 30, 2016 (JP) 2016-068199

(51) Int. Cl. G01N 15/14; C12Q 1/6806; C12Q 1/6825; C12Q 1/686

(52) U.S. Cl. CPC G01N 15/14; C12Q 1/6806; G01N 2015/149; C12Q 1/686; C12Q 1/6825

(57) ABSTRACT

Provided is a highly reliable gene analysis method using a flow cytometry method. The gene analysis method includes a staining step of staining cells, a sorting step of obtaining first information derived from cells in a sample solution by using a flow cytometry method, analyzing the first information according to predetermined extraction conditions, and sorting target cells into a container having arrays of a plurality of wells based on the analyzed results, an amplification step of amplifying DNA of the cells sorted into the container, an analysis step of performing gene analysis on the amplified DNA, and a condition determination step of redetermining the extraction conditions based on at least a piece of information between second information obtained in the amplification step and third information obtained in the analysis step.

### CROSS REFERENCE TO RELATED APPLICATIONS

This application is a Continuation of PCT International Application No. PCT/JP2017/005634 filed on Feb. 16, 2017, which claims priority under 35 U.S.C § 119(a) to Patent Application No. 2016-068199 filed in Japan on Mar. 30, 2016, all of which are hereby expressly incorporated by reference into the present application.

### BACKGROUND OF THE INVENTION

### 1. Field of the Invention

The present invention relates to a gene analysis method.

### 2. Description of the Related Art

A method is known in which target cells are separated and sorted from a sample solution by using a flow cytometry method.

For example, JP2000-157298A describes a method for collecting tiny cells (micronucleus), which are a portion of cells separated due to chromosomal abnormalities, by dividing the cells into main nucleus (parent nucleus) and micronucleus by using a sorting function of a flow cytometer (device used in a flow cytometry method).

### SUMMARY OF THE INVENTION

In the flow cytometry method, the information such as forward-scattered light, side-scattered light, and fluorescence intensity is obtained from cells so as to select cells, and target cells are sorted into a container having a plurality of wells such that one cell is dispensed into one well. The container into which the cells are sorted is set in a Polymerase Chain Reaction (PCR) device, Deoxyribonucleic Acid (DNA) is amplified, and gene analysis is performed.

In the flow cytometry method, target cells are sorted based on the information on fluorescence intensity. Therefore, cells are missorted in some cases due to the nonspecificity of staining, and dead cells cannot be selected in some cases at the time of sorting the cells. Furthermore, in some cases, it is difficult to select cells by using only the information on fluorescence intensity of the flow cytometry method. For example, with the flow cytometry method, nucleated erythrocytes can be selected, but it is difficult to sort nucleated erythrocytes into maternal nucleated erythrocytes and fetal nucleated erythrocytes.

As described above, in a case where target cells cannot be accurately selected by the flow cytometry method, the reliability of the results of the following gene analysis is reduced, and unfortunately, a wrong decision may be made.

The present invention has been made in consideration of the above circumstances, and an object thereof is to provide a highly reliable gene analysis method using a flow cytometry method.

According to an aspect of the present invention, a gene analysis method comprises a staining step of staining cells, a sorting step of obtaining first information derived from cells in a sample solution by using a flow cytometry method, analyzing the first information according to predetermined extraction conditions, and sorting target cells into a container having arrays of a plurality of wells based on the analyzed results, an amplification step of amplifying DNA of the cells sorted into the container, an analysis step of performing gene analysis on the amplified DNA, and a condition determination step of redetermining the extraction conditions based on at least a piece of information between second information obtained in the amplification step and third information obtained in the analysis step.

It is preferable that the staining of the cells is immunostaining by an antigen-antibody reaction.

It is preferable that the first information is at least a piece of information among fluorescence emission, forward-scattered light, and side-scattered light resulting from the immunostaining.

It is preferable that the gene analysis method further comprises an imaging step of imaging the cells sorted into the container between the sorting step and the amplification step, and the extraction conditions are redetermined based on fourth information obtained in the imaging step.

It is preferable that the fourth information includes at least one of the fluorescence intensity, shape, color, and size of the cells.

It is preferable that the second information includes whether or not the DNA is amplified, and the third information includes whether or not the target cells exist.

It is preferable that the amplification step includes a polymerase chain reaction.

It is preferable that the gene analysis is selected from the group consisting of a DNA microarray method, a digital PCR method, a real-time PCR method, a sequencer method, and a combination of these.

It is preferable that the gene analysis method further comprises a concentration step of increasing concentration of the cells in a solvent before the staining step.

According to another aspect of the present invention, a gene analysis method includes a step of staining cells, a sorting step of obtaining first information derived from cells in a sample solution by a flow cytometry method, analyzing the first information based on predetermined extraction conditions, and sorting target cells into a container having arrays of a plurality of wells based on the analyzed results, an imaging step of imaging the cells sorted into the container, an amplification step of amplifying DNA of the cells sorted into the container, an analysis step of performing gene analysis on the amplified DNA, and a condition determination step of redetermining the extraction conditions based on at least a piece of information among second information obtained in the amplification step, third information obtained in the analysis step, and fourth information obtained in the imaging step.

According to the gene analysis method of the present invention, it is possible to realize a highly reliable gene analysis using a flow cytometry method.

### BRIEF DESCRIPTION OF THE DRAWINGS

FIG. 1 is flowchart showing the procedure of a gene analysis method of a first embodiment.

FIG. 2 is a conceptual view of a flow cytometer.

FIG. 3 is a scattergram in which a region including nucleated erythrocytes is selected.

FIG. 4 is a scattergram in which a region where erythrocytes are considered to appear is selected.

FIG. 5 is a scattergram in which a region where nucleated erythrocytes are considered to appear is selected.

FIG. 6 is a perspective view of a container.

FIG. 7 is a perspective view of a container.

FIG. 8 is a scattergram obtained after extraction conditions are redetermined for the scattergram of FIG. 5 based on second information of an amplification step.

FIG. 9 is a scattergram obtained after extraction conditions are redetermined for the scattergram of FIG. 8 based on third information of an analysis step.

FIG. 10 is a flowchart showing the procedure of a gene analysis method of a second embodiment.

FIG. 11 is a view schematically showing the constitution of an image capturing apparatus.

FIG. 12 is a scattergram obtained after extraction conditions are redetermined for the scattergram of FIG. 5 based on fourth information of an imaging step.

FIG. 13 is a scattergram obtained after extraction conditions are redetermined for the scattergram of FIG. 12 based on the second information of the amplification step.

### DESCRIPTION OF THE PREFERRED EMBODIMENTS

Hereinafter, preferred embodiments of the present invention will be described based on the attached drawings. The present invention will be described based on the following preferred embodiments. The present invention can be modified by many techniques without departing from the scope of the present invention, and embodiments other than the above embodiments can be used. Accordingly, all of the modifications in the scope of the present invention are included in claims.

In the drawings, the portions represented by the same references are the same constituents having the same function. Furthermore, in the present specification, in a case where a range of numerical values is represented using “to”, the numerical values as the upper limit and the lower limit represented by “to” are also included in the range of numerical values.

<Gene Analysis Method>

### First Embodiment

A gene analysis method of a first embodiment will be described with reference to drawings. In the present embodiment, the gene analysis method will be described by illustrating a case where blood cells are contained in a sample solution and fetal nucleated erythrocytes are target cells.

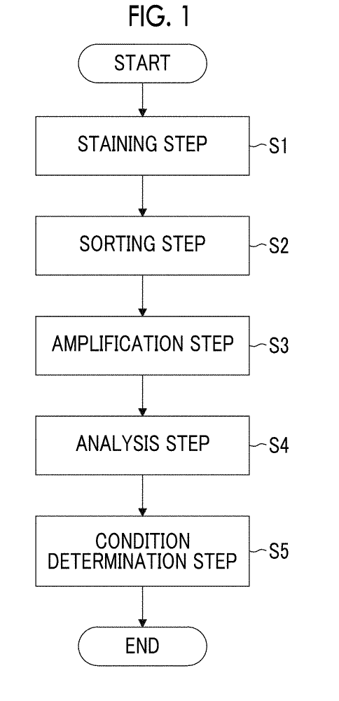

FIG. 1 is a flowchart of the gene analysis method of the first embodiment. As shown in FIG. 1, the gene analysis method includes at least a staining step (step S1), a sorting step (step S2), an amplification step (step S3), an analysis step (step S4), and a condition determination step (step S5).

In the staining step (step S1), cells are stained. In the sorting step (step S2), first information derived from cells in a sample solution is obtained by a flow cytometry method, the first information is analyzed according to predetermined extraction conditions, and target cells are sorted into a container having arrays of a plurality of wells based on the analyzed results. In the amplification step (step S3), DNA of the cells sorted into the container is amplified. In the analysis step (step S4), gene analysis is performed on the amplified DNA. In the condition determination step (step S5), based on at least a piece of information between second information obtained in the amplification step and third information obtained in the analysis step, the extraction conditions are redetermined. Hereinafter, each of the steps will be described.

<Staining Step (Step S1)>

In the present embodiment, the gene analysis method includes a step of staining cells. The staining of cells makes it possible to obtain the first information derived from the cells in the sample solution by a flow cytometry method which will be described later.

The staining of cells is preferably immunostaining by an antigen-antibody reaction. The antigen-antibody reaction refers to a reaction in which an antibody specifically binds to an antigen having a complementary structure, and the immunostaining means a technique of causing a fluorescent dye-conjugated antibody to bind to an antigen present in a cell.

The immunostaining includes a direct method and an indirect method. The direct method is a method of directly conjugating a fluorescent dye to an antibody and causing the antibody to react with an antigen. In contrast, the indirect method is a method of conjugating a fluorescent dye not to an antibody (primary antibody) which can specifically bind to an antigen which should be detected but to an antibody (secondary antibody) which can specifically bind to the primary antibody so as to detect the antigen.

Examples of the antibody that makes cells immunostained by the antigen-antibody reaction include anti-human CD antibodies such as an anti-CD3 antibody, an anti-CD4 antibody, an anti-CD14 antibody, an anti-CD25 antibody, an anti-CD45 antibody, an anti-CD71 antibody, and an anti-CD127 antibody. Examples of the fluorescent dye include 4′,6-diamidine-2′-phenylindole dihydrochloride (DAPI: 4′,6-diamidino-2-phenylindole), propidium iodide (PI), Pyronin Y, fluorescein isothiocvanate (FITC), phycoerythrin (PE), allophycocyanin (APC), Texas Red (TR (registered trademark)), Hoechst 33342, 7-aminoactinomycin D (7-AAD), 2′-deoxycytidine 5′-triphosphoric acid (Cy3), sulfoindocyanine succinimidyl ester (Cy5), DRAQ5 (registered trademark), Brilliant Violet 570, Brilliant Violet 421, and the like.

The sample solution is prepared as below. First, a sample to be analyzed containing target cells is prepared. The sample to be analyzed is mixed, for example, with antibodies conjugated to a fluorescent dye used for immunostaining and incubated, thereby immunostaining the cells. In this way, a sample solution is prepared which contains cells immunostained by an antigen-antibody reaction.

<Sorting Step (Step S2)>

In the sorting step, first information derived from the cells in the sample solution is obtained using a flow cytometer 10 performing a flow cytometry method, the first information is analyzed according to predetermined extraction conditions, and target cells are sorted into a container having arrays of a plurality of wells based on the analyzed results.

The extraction conditions are determined, for example, based on the knowledge of the past by seeing the distribution in a scattergram.

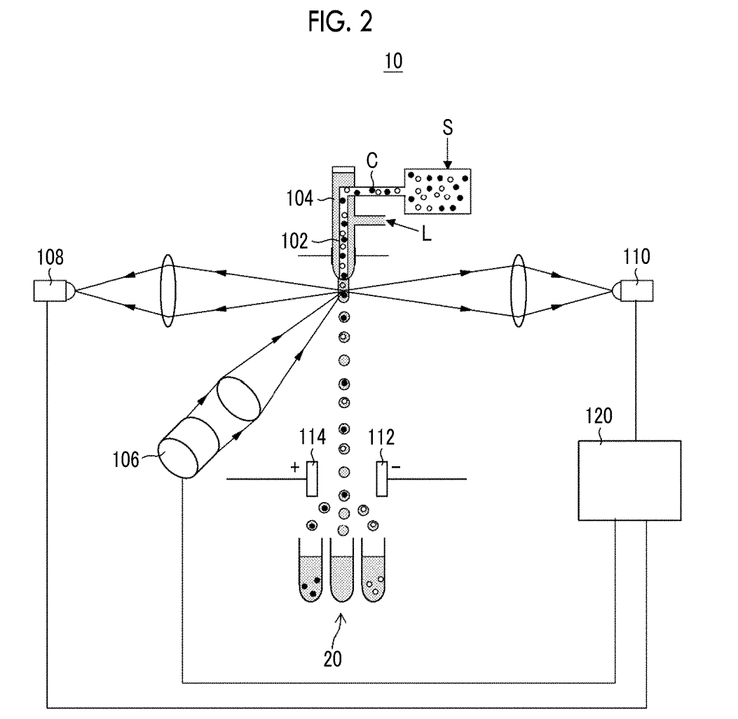

FIG. 2 is a conceptual view of the flow cytometer 10. A sample solution S contains blood cells including cells C immunostained by an antigen-antibody reaction.

The sample solution S is introduced into a flow cell 104 from a nozzle 102. A sheath liquid L is introduced into the flow cell 104. In the flow cell 104, the sample solution S is squeezed by the sheath liquid L. Because the sample solution S is squeezed, the cells C are arrayed in a line.

The cells C are irradiated, for example, with laser beams from a light source 106. By the irradiation of the laser beams, the immunostaining of the cells C is excited, and the cells C emit fluorescence by the immunostaining. The fluorescence intensity of the emitted fluorescence is detected by a detector 108. The fluorescence emitted from the cells C that is detected by the detector 108 is obtained as first information derived from cells, and input and stored in a controller 120. The controller 120 includes an operation unit performing various processes, various programs, a storage unit storing data, and the like.

Laser beams are radiated from the light source 106, and forward-scattered light and side-scattered light emitted from the cells C by the immunostaining are detected by a detector 110. The fluorescence intensity by the forward-scattered light and the side-scattered light from the cells C detected by the detector 110 is obtained as the first information derived from cells, and input and stored in the controller 120.

Hitherto, a case has been illustrated in which the information on the fluorescence emission, the forward-scattered light, and the side-scattered light resulting from the immunostaining is obtained as the first information. However, as the first information, at least a piece of information among the fluorescence emission, the forward-scattered light, and the side-scattered light resulting from the immunostaining may be obtained.

The size of a cell to be measured is measured by the forward-scattered light obtained as the first information, and the structure of a cell to be measured or the like is measured by the side-scattered light and the fluorescence emission.

Ultrasonic waves are applied to the flow cell 104, and hence liquid droplets containing the cell C are formed. Based on the results of the detection described above, the controller 120 causes the liquid droplets to be negatively or positively charged. The controller 120 does not cause liquid droplets, which will be discarded, to be charged. At the time of passing through deflection electrode plates 112 and 114, the charged liquid droplets are attracted to any of the deflection electrode plates 112 and 114. As a result, basically, one cell is sorted into one well in the container 20.

As the light source 106 exciting the immunostaining, a plurality of laser light sources having different wavelengths are preferably used. For example, it is preferable that the flow cytometer includes a laser light source having a wavelength of 405 nm, a laser light source having a wavelength of 488 nm, a laser light source having a wavelength of 561 nm, and a laser light source having a wavelength of 683 nm. In a case where a plurality of laser light sources having different wavelengths are used, a plurality of fluorescence intensities can be obtained as the first information derived from cells.

Furthermore, it is preferable to use a fluorescence filter which cuts the excitation light of laser light sources for simultaneously detecting fluorescence intensities and selectively transmits the wavelength of light emitted from a fluorescent dye by immunostaining.

The controller 120 of the flow cytometer 10 stores an analysis program for analyzing detection results based on the first information (the fluorescence emission, the forward-scattered light, and the side-scattered light resulting from immunostaining) derived from cells. The controller 120 obtained the first information derived from cells and analyzes the first information according to predetermined extraction conditions.

For example, based on the first information derived from cells, the controller 120 can create a scattergram (scatter plot) in which any of the fluorescence emission, the forward-scattered light, and the side-scattered light is plotted on the ordinate or the abscissa. By creating the scattergram from the first information derived from cells, all of the detected cells can be divided into a plurality of groups so as to sort target cells. Furthermore, on the scattergram created from the first information, by specifying a region (so-called gating) or by excluding certain cells from a specified region (so-called gating out), a group can be separated from all cells or from other groups on the graph, and the data can be narrowed down to a group including target cells.

In the present embodiment, the analysis of the first information includes a series of processes for narrowing the data down to a group including target cells from the first information derived from cells, and the extraction conditions can be determined by appropriately combining the selection of the ordination or the abscissa for creating the scattergram, the gating and gating out for separating a group from other groups, and the like.

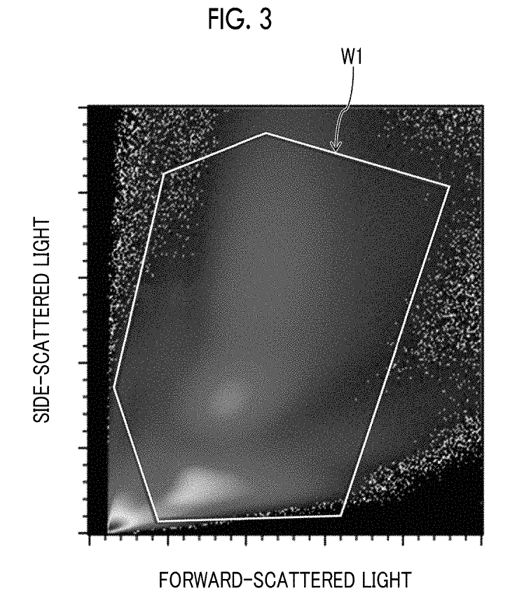

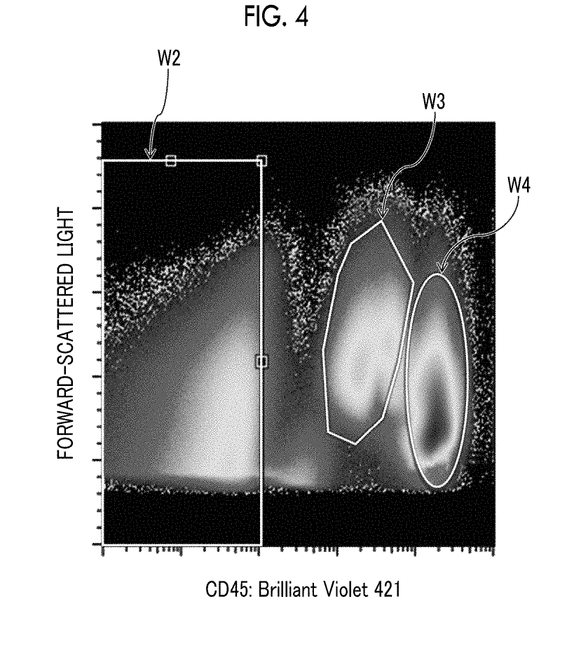

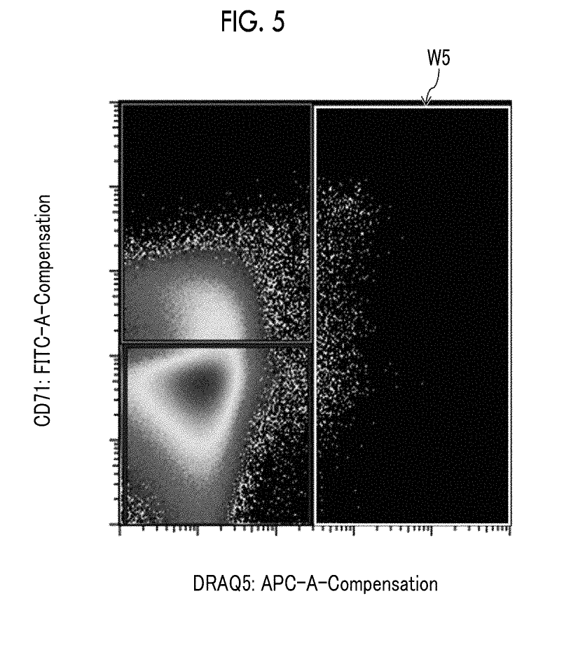

FIG. 3 to FIG. 5 show an example of a case where the first information is analyzed according to the predetermined extraction conditions. FIG. 3 is a scattergram in which a region including nucleated erythrocytes is selected. FIG. 4 is a scattergram in which a region where erythrocytes appear is selected. FIG. 5 is a scattergram in which a region where nucleated erythrocytes appear is selected.

FIG. 3 is a scattergram in which the fluorescence intensity of the side-scattered light is plotted on the ordinate and the fluorescence intensity of forward-scattered light is plotted on the abscissa. The scattergram of FIG. 3 shows all cells which pass through the flow cell and from which the first information is obtained. In FIG. 3, while a region W1 considered to include nucleated erythrocytes is selected by gating, platelets are excluded from the region W1. By the gating, the group of the region W1 including nucleated erythrocytes is separated from all cells.

FIG. 4 is a scattergram which is targeted at the group of the region W1 selected in FIG. 3 and in which the fluorescence intensity of the forward-scattered light is plotted on the ordinate and the fluorescence intensity of CD45:Brilliant Violet 421 is plotted on the abscissa. CD45 is a leukocyte common antigen, and the leukocyte is immunostained with Brilliant Violet 421. Accordingly, by gating CD45 negativity, a region W2 in which erythrocytes are considered to appear is selected. The group in which erythrocytes are considered to appear is separated from other groups. A region W3 is a group in which granulocytes are considered to appear, and a region W4 is a group in which lymphocytes and monocytes are considered to appear.

FIG. 5 is a scattergram which is targeted at the group in the region W2 selected in FIG. 4 and in which the fluorescence intensity of CD71:FITC is plotted on the ordinate and the fluorescence intensity of DRAQ5:APC is plotted on the abscissa. The fluorescence intensity of CD71:FITC is correlated with the juvenility of erythrocytes, and the fluorescence intensity of DRAQ5:APC is correlated with the nucleus. Accordingly, by gating DRAQ5:APC positivity, a region W5 in which nucleated erythrocytes are considered to appear is selected. The group in which nucleated erythrocytes are considered to appear is separated from other groups.

In the present embodiment, the group, which is selected from the region W5 based on the results of the analysis described above and in which nucleated erythrocytes are considered to appear, is sorted into the container 20 by the flow cytometer 10.

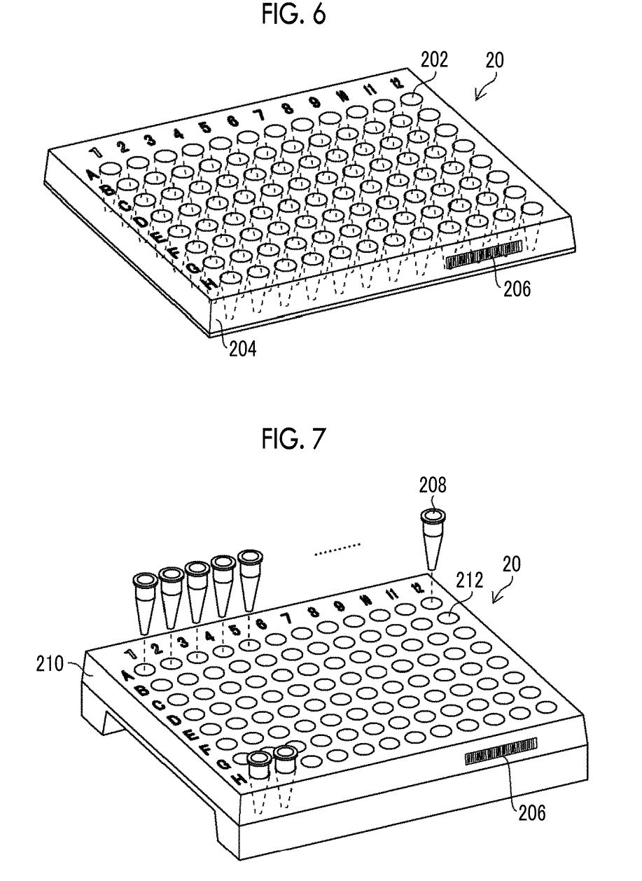

Next, the container 20 into which the cells are sorted will be described. FIGS. 6 and 7 are perspective views of the container 20.

As shown in FIG. 6, the container 20 has a plurality of wells 202 each having an opening and a bottom surface for collecting a plurality of cells and side walls 204 forming an integral structure with the plurality of wells 202. The plurality of wells 202 are arrayed in rows and columns. In order to identify the position of each of the wells 202, numbers representing the rows and alphabets representing the columns are marked on the opening side of the wells 202 of the container 20. In the container 20 shown in FIG. 6, cells are collected into each of the wells 202. In order to identify the container 20, for example, an identification label 206 such as a bar code is marked on the side wall of the container 20.

As shown in FIG. 7, a container 20 having a shape different from that shown in FIG. 6 has a plurality of tubes 208 each having an opening and a bottom surface for collecting a plurality of cells and a supporting member 210 including a plurality of holes 212 for holding the plurality of tubes 208. In the container 20 shown in FIG. 7, the tubes 208 function as wells. As long as the wells each have an opening and a bottom surface for accommodating cells, the shape of the wells and the like are not limited.

The plurality of holes 212 form rows and columns. In order to identify the position of each of the holes 212, numbers representing the rows and alphabets representing the columns are marked on the side, on which the holes 212 are formed, of the container 20. In the container 20 shown in FIG. 7, cells are collected into each of the tubes 208 held in the supporting member 210. Furthermore, in order to identify the container 20, for example, the identification label 206 such as a bar code is marked on the side wall of the supporting member 210. The tubes 208 may be constituted with individual unit tubes or may be constituted with a plurality of tubes connected to each other. In addition, each of the tubes 208 may have a cap (not shown in the drawing).

As described above, by the flow cytometer 10, the cells in the selected region W5 are sorted as target cells into the wells 202 of the container 20 or the tubes 208 of the container 20 basically as a single cell unit.

The controller 120 preferably stores the positions (wells 202 or tubes 208) in the container 20 accommodating cells and the first information derived from the cells by correlating the positions with the information. The positions in the container 20 accommodating the cells are preferably identified by the rows and columns marked in the container 20 and the identification label 206.

<Amplification Step (Step S3)>

In the amplification step. DNA of the sorted cells in the container is amplified. It is preferable that the amplification step includes a polymerase chain reaction. Hereinafter, the amplification step will be described by illustrating the polymerase chain reaction (PCR).

The container 20 containing the sorted cells is set in a PCR device. The container set in the PCR device may be the container 20 containing cells sorted using the flow cytometer 10 or a container for PCR to which the cells are moved from the container 20. The sorted cells in the container mean cells sorted into the container in the sorting step, and do not mean that the container of the sorting step is used as long as these cells are amplified in the amplification step. In a case where the container set in the PCR device is different from the container 20 used in the flow cytometer 10, the first information derived from cells in the sorting step and the positional information of the container 20 containing sorted cells are stored, for example, in the controller 120 by being correlated with the container set in the PCR device.

As a first step, in the PCR device, the reaction solution is heated to about 94° C. and then kept at the same temperature for 30 seconds to 1 minute such that the double-stranded DNA splits and becomes a single strand. As a second step, the reaction solution is rapidly cooled to about 60° C., the sing-stranded DNA and a primer are heated (annealed) to a predetermined temperature, and the single-stranded DNA and the primer are heated. As a third step, a DNA polymerase is reacted with the primer, and the reaction solution is heated to a temperature (about 60° C. to 72° C.) which is suitable for the DNA polymerase activity but does not cause the separation between the single-stranded DNA and the primer. This state is maintained for a time taken for DNA synthesis (the time varies with the length of DNA amplified, but is generally 1 to 2 minutes).

The first to third steps are regarded as one cycle, and by performing a plurality of cycles, for example, 20 cycles, a specific DNA fragment can be amplified. Generally, provided that n cycles of the PCR process are performed, from one double-stranded DNA, a target portion can be amplified by a factor of 2n.

The aforementioned amplification procedure is illustrated as an example of the polymerase chain reaction, and the amplification step is not limited thereto.

In the amplification step, the second information correlated with the results of the amplification is obtained. For example, as the second information, whether or not DNA of target cells has been amplified, that is, whether or not amplification has occurred is preferably obtained. Based on whether or not the amplification has occurred, whether the sorted cells are living cells or dead cells can be decided. The first information derived from cells and the second information obtained in the amplification step are correlated with each other, and input and stored, for example, in the controller 120.

The second information about whether or not DNA has amplified can be obtained preferably by performing electrophoresis on the DNA fragment by using agarose gel. By the electrophoresis, it is possible to check whether or not DNA exists or to check whether or not DNA has amplified based on the size of DNA.

<Analysis Step (Step S4)>

In the analysis step, gene analysis is performed on the amplified DNA. The gene analysis is selected from the group consisting of a DNA microarray method, a digital PCR method, a real-time PCR method, a sequencer method, and a combination of these. The gene analysis is not particularly limited, but nCounter System (manufactured by NanoString Technologies, Inc.) can be used. In the present embodiment, in view of the accuracy and speed of the analysis, the number of samples that can be treated at a time, and the like, it is preferable to use a so-called next-generation sequencer method.

The DNA microarray method is a method of arraying DNA fragments of cells on a substrate at high density, performing hybridization on the DNA arrays on the substrate, and analyzing the genetic information expressed in the cells.

The digital PCR method is a method of distributing a target sample into a plurality of wells, performing individual PCR processes in parallel, and counting the number of positive reactions at the end of amplification.

In the present embodiment, the next-generation sequencer means a sequence classified as a sequencer contrasted with a capillary sequencer (referred to as a first-generation sequencer) using the Sanger's method. The next-generation sequencer includes a second generation, a third generation, and a fourth generation. Currently, the most widespread next-generation sequencer is a sequencer using a principle of determining a base sequence by measuring fluorescence or luminescence related with the binding of a complementary strand by a DNA polymerase or the synthesis of a complementary strand by a DNA ligase.

Specifically, examples thereof include MiSeq (manufactured by Illumina, Inc.), HiSeq 2000 (manufactured by Illumina, Inc., HiSeq is a registered trademark), Roche 454 (manufactured by Hoffmann-La Roche Ltd), and the like.

In a case where the DNA amplification product obtained by the amplification step is analyzed using the next-generation sequencer, it is possible to use whole genome sequencing, exome sequencing, and amplicon sequencing.

Examples of means for aligning sequence data obtained by the next-generation sequencer include Burrows-Wheeler Aligner (BWA). It is preferable to map the sequence data to a known human genome sequence by using BWA. Examples of means for analyzing genes include SAMtools and BEDtools. It is preferable to analyze gene polymorphism, gene variant, and the number of chromosomes by using the analysis means.

By performing gene analysis on the amplified DNA, third information correlated with the results of the gene analysis is obtained. For example, as the third information, whether or not the sorted cells are target cells, that is, whether or not target cells exist is preferably obtained. The first information derived from cells and the third information obtained in the analysis step are input and stored in, for example, the controller 120 by being correlated with each other.

<Condition Determination Step (Step S5)>

In the condition determination step, based on at least a piece of information between the second information obtained in the amplification step and the third information obtained in the analysis step, the extraction conditions in the sorting step are redetermined.

The redetermination of the extraction conditions is newly determining the extraction conditions predetermined in the sorting step, and includes a case where the extraction conditions predetermined in the sorting step are changed and a case where the extraction conditions predetermined in the sorting step are not changed. The extraction conditions include selecting the ordinate or the abscissa for creating a scattergram or appropriately combining gating, gating out, and the like for separating a group from other groups.

In the sorting step, for example, the first information including at least one of the fluorescence emission, the forward-scattered light, or the side-scattered light is obtained resulting from immunostaining, the first information is analyzed according to the predetermined extraction conditions, and cells considered as target cells are sorted based on the analyzed results. Therefore, generally, with only the first information, it is difficult to perform sorting by separating non-target cells (for example, dead cells) and to sort cells and the like that are difficult to be classified into a plurality of groups.

In the present embodiment, after the sorting step, at least one of the second information or the third information is obtained in the amplification step and the analysis step by being correlated with the first information. Through the amplification step and the analysis step, it is possible to decide whether or not the sorted cells are target cells. Preferably, because the second information includes information on whether or not the gene has been amplified, it is possible to decide whether the sorted cells are living cells or dead cells. Furthermore, because the third information includes genetic information, it is possible to decide whether the sorted cells are target cells.

At least one of the second information or the third information is correlated with the first information. Therefore, based on the second information and the third information, the extraction conditions predetermined in the sorting step can be provided with feedback. Accordingly, it is possible to redetermine extraction conditions that enable the target cells to be sorted in a higher probability.

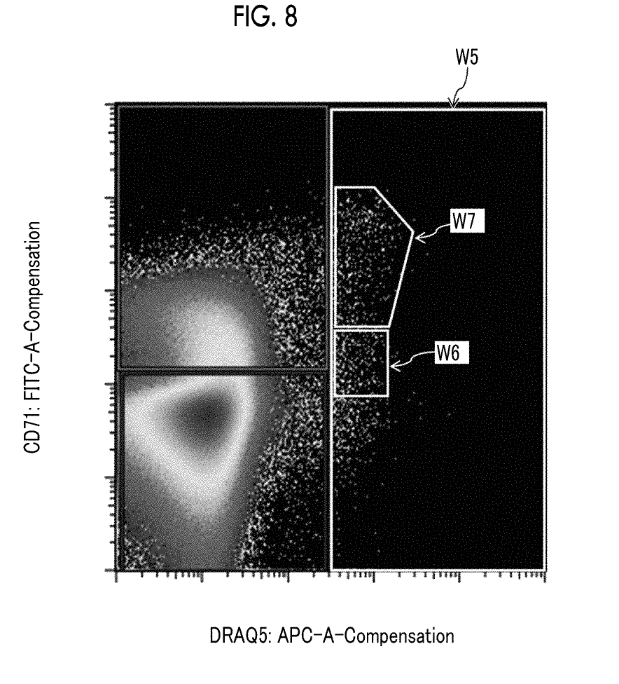

FIG. 8 is a scattergram obtained after redetermining the extraction conditions for the scattergram of FIG. 5 based on the second information of the amplification step. The first information correlated with the second information obtained in the amplification step is provided as feedback, for example, to the controller 120 of the flow cytometer 10. The controller 120 can redetermine the extraction conditions based on the second information receiving the feedback.

As a result of analyzing the first information based on the redetermined extraction conditions, a region W6 is newly selected. The region W6 reflecting the results based on the second information in the region W5 is assumed to be a region in which a lot of cells not being amplified (dead cells) may appear. By gating the region W6 out, a new region W7 is selected. It is assumed that in the region W7, a lot of nucleated erythrocytes which are living cells may appear.

It is understood that by redetermining the extraction conditions based on the second information, the region W7, in which a lot of nucleated erythrocytes as living cells appear, can be separated from other groups in a high probability.

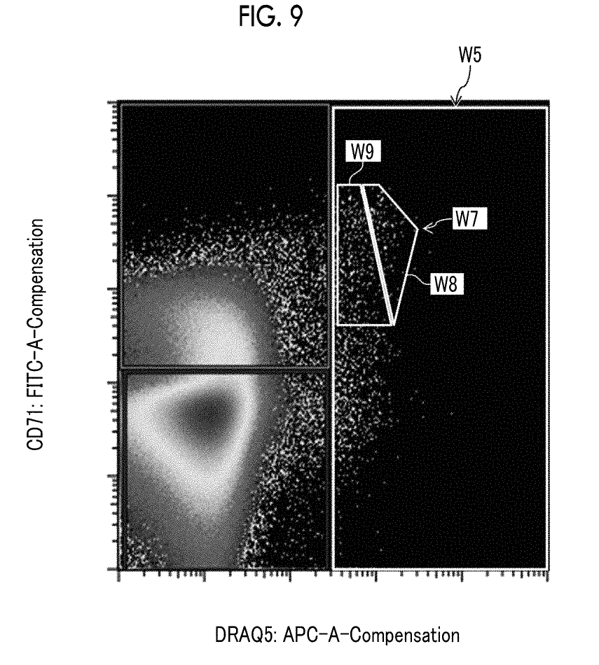

FIG. 9 is a scattergram obtained after redetermining the extraction conditions for the scattergram of FIG. 8 based on the third information of the analysis step. The first information correlated with the third information obtained in the analysis step is provided as feedback, for example, to the controller 120 of the flow cytometer 10. The controller 120 can redetermine the extraction conditions based on the third information receiving the feedback.

As a result of analyzing the first information based on the redetermined extraction conditions, a region W8 and a region W9 are newly selected. It is assumed that the region W8 reflecting the results based on the third information in the region W7 is a region in which a lot of maternal nucleated erythrocytes may appear, and the region W9 is a region in which a lot of fetal nucleated erythrocytes may appear. It is understood that by redetermining extraction conditions based on the third information, the region W9, in which many target cells (in a case where the target cells are nucleated erythrocytes derived from a fetus) appear, can be separated from other groups in a high probability.

In the present embodiment, a case where the extraction conditions are redetermined based on the second information and the third information has been described. However, the extraction conditions may be redetermined based on at least a piece of information between the second information and the third information. In a case where the extraction conditions are redetermined based on at least a piece of information, target cells can be sorted in a higher probability, and accordingly, a highly reliable gene analysis method can be realized.

### Second Embodiment

Next, a gene analysis method of a second embodiment will be described with reference to drawings. In the present embodiment, the gene analysis method will be described by illustrating a case where blood cells are contained in a sample solution and fetal nucleated erythrocytes are target cells.

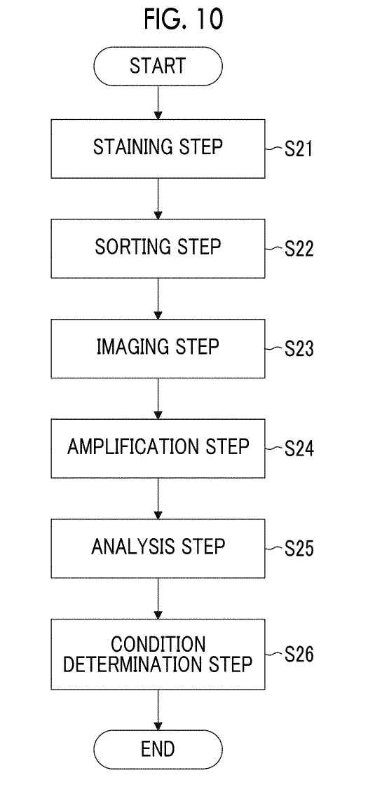

FIG. 10 is a flowchart of the gene analysis method of the second embodiment. As shown in FIG. 10, the gene analysis method includes at least a staining step (step S21), a sorting step (step S22), an imaging step (step S23), an amplification step (step S24), an analysis step (step S25), and a condition determination step (step S26).

In the staining step (step S21), cells are stained. In the sorting step (step S22), first information derived from cells in a sample solution is obtained by a flow cytometry method, the first information is analyzed according to predetermined extraction conditions, and target cells are sorted into a container having arrays of a plurality of wells based on the analyzed results. In the imaging step (step S23), the cells sorted into the container are imaged. In the amplification step (step S24), DNA of the cells sorted into the container is amplified. In the analysis step (step S25), gene analysis is performed on the amplified DNA. In the condition determination step (step S26), based on at least a piece of information among second information obtained in the amplification step, third information obtained in the analysis step, and fourth information obtained in the imaging step, extraction conditions are redetermined.

Hereinafter, each of the steps will be described. In the following section, the same steps as those in the first embodiment will not be described in some cases.

As the staining step (step S21) and the sorting step (step S22) of the gene analysis method of the second embodiment, the same staining steps (step S1) and sorting step (step S2) as those in the first embodiment can be performed. Next, the imaging step (step S23) will be described.

<Imaging Step (Step S23)>

In the imaging step, the cells sorted into the container are imaged. Imaging cells means that the image of the cells are captured, and includes a case where target cells, non-target cells, or foreign substances (dust or cell fragments) that are not cells are imaged. In the imaging step, it is preferable to use an image capturing apparatus 30 for imaging the cells sorted into the container 20. Examples of the image capturing apparatus 30 include a fluorescence microscope including an imaging device.

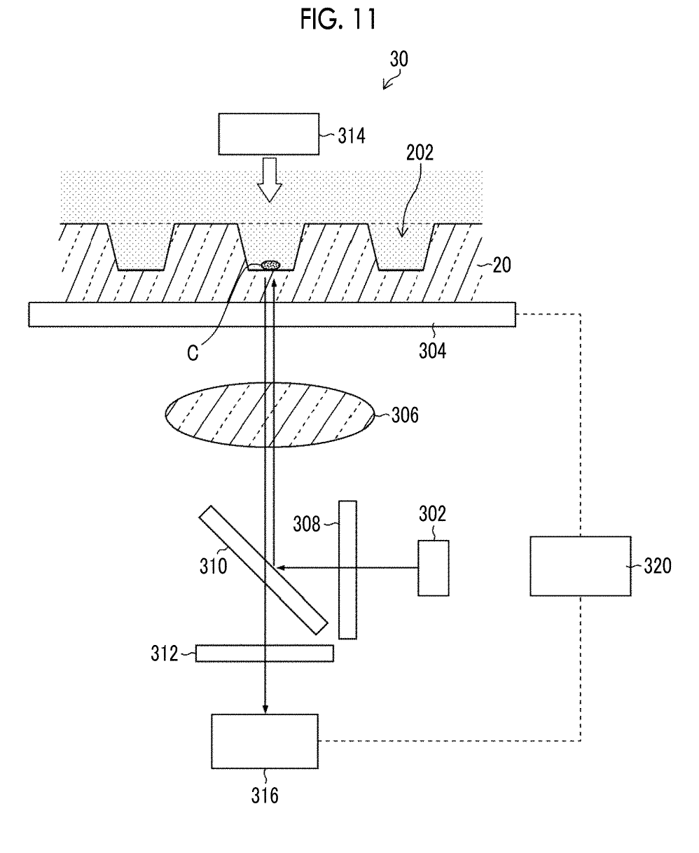

FIG. 11 is a view schematically showing the constitution of the image capturing apparatus 30. The image capturing apparatus 30 can image cells C collected into the container 20. The image capturing apparatus 30 is constituted such that fourth information derived from cells can be obtained by imaging the cell C. The fourth information derived from cells include at least one of the fluorescence intensity from the cell, the cell shape, the cell color, or the cell size. The fluorescence intensity means fluorescence emitted from a fluorescent dye resulting from immunostaining excited by excitation light. The cell shape includes the external and internal forms of the cell. The cell color means the color of the cell. The cell size includes an area obtained by two-dimensional observing the cell, a volume obtained by three-dimensionally observing the cell, and the like.

In the present embodiment, a case will be described where the cells sorted into the container 20 are imaged from a side opposite to the opening (that is, a rear surface) of the wells 202 of the container 20 in the imaging step.

The image capturing apparatus 30 includes a first light source 302 for excitation that is for measuring fluorescence of the cell C, a table 304 for loading the container 20, a lens 306 spaced apart from the table 304 and disposed on a side opposite to the container 20, a filter group constituted with an excitation filter 308, a dichroic mirror 310, and a fluorescent filter 312, a second light source 314 which is disposed on the side of the well 202 of the container 20 and irradiates the container 20 with light for measuring transmitted light, and an imaging device 316 imaging the cell C.

The imaging device 316 is disposed on the side opposite to the opening (front surface) of the well 202 of the container 20 into which the cell C is sorted. That is, the imaging device 316 can image the cell C from the rear surface of the container 20. The excitation light from the first light source 302 is radiated to the well 202 from the rear surface of the container 20, and the light from the second light source 314 is radiated to the well 202 from the front surface of the container 20.

In order for the container 20 to be irradiated with the excitation light from the rear surface side thereof or in order to transmit light and receive fluorescence from the cell and receive transmitted light, it is preferable that the material of the container 20 is transparent, is not autofluorescent, and does not scatter light.

Preferably, the image capturing apparatus 30 can obtain images by imaging the cell C emitting fluorescence and images by imaging the cell C in a bright field.

As the first light source 302, for example, it is possible to use a high-pressure mercury lamp, a high-pressure xenon lamp, a light emitting diode, a laser diode, a tungsten lamp, a halogen lamp, a white light emitting diode, and the like. Even in a case where these light sources are used, only a target wavelength can be transmitted through the excitation filter 308. The fluorescent dye of the immunostained cell C can be irradiated with light having a target excitation wavelength. As the second light source 314, the same light source as the first light source 302 can be used.

A case where the fluorescence intensity of the cell C resulting from immunostaining is obtained as an image by the imaging device 316 will be described. Among the lights radiated from the first light source 302, only the light in a target wavelength range is transmitted through the excitation filter 308. The light transmitted through the excitation filter 308 is reflected toward the container 20 by the dichroic mirror 310. The light reflected by the dichroic mirror 310 is transmitted through the lens 306 and radiated to the cell C collected in the well 202. The light radiated to the cell C is in a wavelength range exciting the fluorescent dye of the immunostained cell C. The immunostained cell C is excited by the excitation light and emits fluorescence of a wavelength different from the excitation wavelength radiated. The fluorescence of the cell C resulting from immunostaining passes through the lens 306, the dichroic mirror 310, and the fluorescent filter 312 and imaged by the imaging device 316, and in this way, an image is obtained. The wavelength of the fluorescence emitted by the excitation light is longer than the wavelength of the excitation light. Therefore, by the dichroic mirror 310, the light of the wavelength of the excitation light can be reflected toward the container 20 side, and the light of the wavelength of the fluorescence can be transmitted toward the imaging device 316 side. Furthermore, the fluorescent filter 312 can transmit only the fluorescence without transmitting the excitation light. Accordingly, in the imaging device 316, the cell C emitting fluorescence by immunostaining can be imaged. Because the fluorescent filter 312 transmits only the fluorescence, the image captured by the imaging device 316 is not affected by the excitation light. Consequently, an accurate image can be obtained.

The image capturing apparatus 30 of the present embodiment has the table 304 and a driving device (not shown in the drawing) for moving the container 20 to any position (for example, in the X direction, the Y direction, or the Z direction). By the table 304 and the driving device, a specific well 202 in the container 20 can be moved to an observation position. It is preferable that the driving device can move the table 304 in the X direction, the Y direction, and the Z direction.

In a case where the cell C is immunostained with a plurality of fluorescent dyes, by the switching between different filter groups (the excitation filter 308, the dichroic mirror 310, and the fluorescent filter 312), the cell C emitting different types of fluorescence can be imaged, and the image of the cell C can be obtained.

The imaging device 316 is not particularly limited as long as it can image the fluorescence of the cells in the wells 202 of the container 20 or can image the transmitted light. As the imaging device 316, for example, a charge-coupled device (CCD) camera can be used.

In the present embodiment, the image capturing apparatus 30 has been described in which the imaging device 316, the first light source 302, and the filter group are disposed on the rear surface side of the container 20 while the second light source 314 is disposed on the front surface side of the container. The present invention is not limited thereto, and an image capturing apparatus 30 can also be used in which the imaging device 316, the first light source 302, and the filter group are disposed on the front surface side of the container 20 while the second light source 314 is disposed on the rear surface side of the container.

The fourth information obtained by imaging the cell in the imaging step is correlated with the first information derived from cells and then input and stored, for example, in the controller 120 of the flow cytometer 10. Because the image prepared by imaging the cell C is obtained in the imaging step, as the fourth information, information including at least one of the fluorescence intensity, shape, color, or size of the cell can be obtained from the image. Because the fourth information is obtained by directly observing the cell in the imaging step, it is possible to decide whether or not the sorted cells are target cells, non-target cells, dust, or cell fragments.

In the present embodiment, a case where the container 20 shown in FIG. 6 is used has been described. However, the present invention is not limited thereto, and a container 20 shown in FIG. 7 can also be used for imaging cells.

As the amplification step (step S24) and the analysis step (step S25) of the gene analysis method of the second embodiment, the same amplification step (step S3) and analysis step (step S4) as those in the first embodiment can be performed. Next, the condition determination step (step S26) will be described.

<Condition Determination Step (Step S26)>

In the condition determination step, based on at least a piece of information among the second information obtained in the amplification step, the third information obtained in the analysis step, and the fourth information obtained in the imaging step, the extraction conditions in the sorting step are redetermined.

In the present embodiment, after the sorting step, at least one of the second information, the third information, or the fourth information is obtained in the amplification step, the analysis step, and the imaging step by being correlated with the first information. Through the amplification step, the analysis step, and the imaging step, it is possible to decide whether or not the sorted cells are target cells. Preferably, the second information includes whether or not the gene has been amplified, and accordingly, it is possible to decide whether the sorted cells are living cells or dead cells. In addition, because the third information includes genetic information, it is possible to decide whether the sorted cells are target cells. Furthermore, because the fourth information includes the fluorescence intensity of the cells and the like, it is possible to decide whether the sorted cells are target cells from the captured image.

At least one of the second information, the third information, or the fourth information is correlated with the first information. Therefore, based on the second information, the third information, and the fourth information, feedback can be provided to the extraction conditions in the sorting step. Accordingly, it is possible to redetermine the extraction conditions that enable target cells to be sorted in a higher probability.

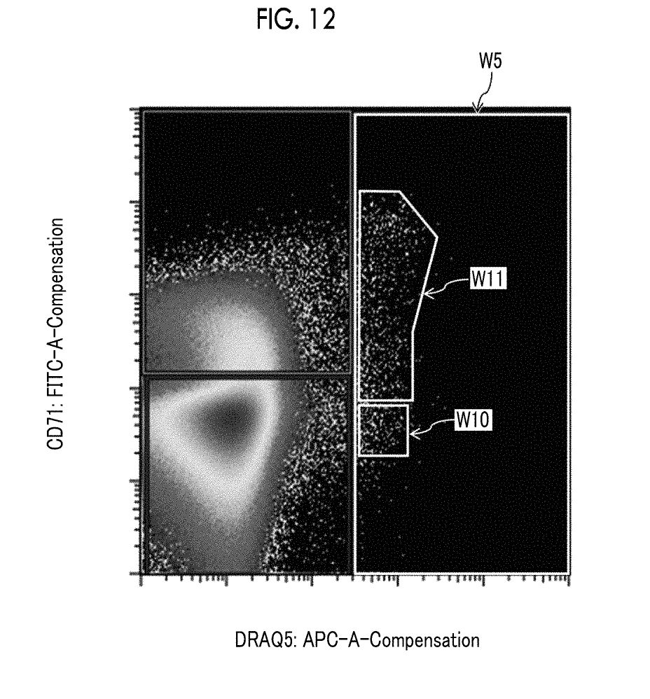

FIG. 12 is a scattergram obtained after redetermining the extraction conditions for the scattergram of FIG. 5 based on the fourth information of the imaging step. The first information correlated with the fourth information obtained in the imaging step is provided as feedback, for example to the controller 120 of the flow cytometer 10. The controller 120 can redetermine the extraction conditions based on the fourth information receiving the feedback.

As a result of analyzing the first information based on the redetermined extraction conditions, a region W10 is newly selected. The region W10 reflecting the results based on the fourth information in the region W5 is assumed to be a region in which a lot of dust and cell fragments may appear. By gating the region W10 out, a new region W11 is selected. The region W11 is assumed to be a region in which a lot of nucleated erythrocytes may appear except for dust or cell fragments.

It is understood that by redetermining the extraction conditions based on the fourth information, the region W11, in which a lot of target cells appear except for dust, cell fragments, and the like appear, can be separated from other groups in a high probability.

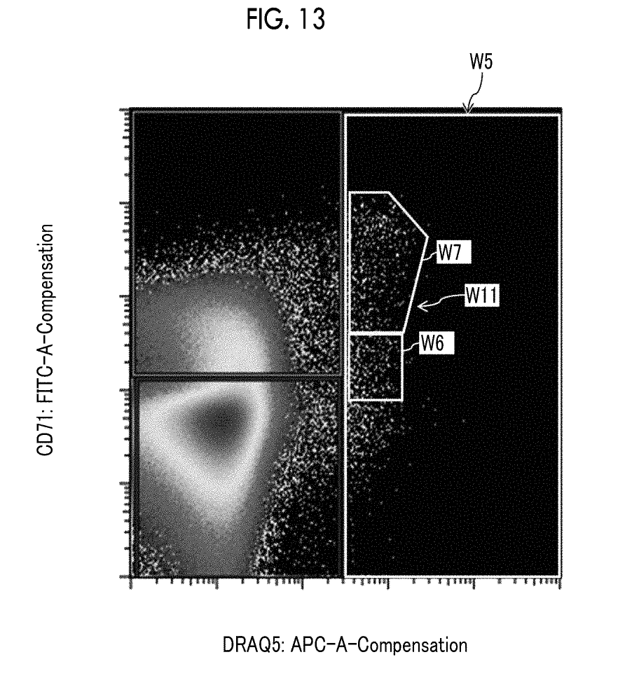

FIG. 13 is a scattergram obtained after redetermining the extraction conditions for the scattergram of FIG. 12 based on the second information of the amplification step. The first information correlated with the second information obtained in the amplification step is provided as feedback, for example, to the controller 120 of the flow cytometer 10. The controller 120 can redetermine the extraction conditions based on the second information receiving the feedback.

As a result of analyzing the first information based on the redetermined extraction conditions, a region W6 is newly selected. The region W6 reflecting the results based on the second information in the region W11 is assumed to be a region in which a lot of cells (dead cells) not being amplified may appear. By gating the region W6 out, a new region W7 is selected in the region W11. It is assumed that a lot of nucleated erythrocytes which are living cells may appear in the region W7.

It is understood that by redetermining the extraction conditions based on the second information, the region W7, in which a lot of nucleated erythrocytes which are living cells appear, can be separated from other groups in a high probability.

The first information correlated with the third information obtained in the analysis step is provided as feedback, for example, to the controller 120 of the flow cytometer 10. The controller 120 can redetermine the extraction conditions based on the third information receiving the feedback.

Consequently, as shown in the scattergram of FIG. 9, as a result of analyzing the first information based on the redetermined extraction conditions, a region W8 and a region W9 are newly selected.

The region W8 reflecting the results based on the third information in the region W7 is assumed to be a region in which a lot of maternal nucleated erythrocytes may appear, and the region W9 is assumed to be a region in which a lot of fetal nucleated erythrocytes may appear. It is understood that by redetermining the extraction conditions based on the third information, the region W9, in which a lot of target cells (a case where the target cells are fetal nucleated erythrocytes) appear, can be separated from other groups in a high probability.

In the present embodiment, a case where the extraction conditions are redetermined based on the second information, the third information, and the fourth information has been described. However, the extraction conditions may be redetermined based on at least a piece of information among the second information, the third information, and the fourth information. In a case where the extraction conditions are redetermined based on at least a piece of information, target cells can be sorted in a higher probability. Accordingly, a highly reliable gene analysis method can be realized.

In the present embodiment, it is preferable that the gene analysis method includes, before the staining step, a concentration step of increasing the concentration of target cells in a solvent. The concentration step will be described by illustrating a case where nucleated erythrocytes in the maternal blood are concentrated as an example.

<Concentration Step>

It is preferable that the nucleated erythrocytes in the maternal blood are concentrated before the staining step such that the density of the nucleated erythrocytes is increased. As the concentration step, it is possible to use a known method such as a density gradient centrifugation method, a magnetic activated cell sorting (MACS) method, a fluorescence activated cell sorting (FACS) method, a lectin method, or a filtration method. Among these, as a simple concentration method exploiting the characteristics of blood cells, a density gradient centrifugation method is preferably used for performing concentration. Hereinafter, as an example of the concentration step, the density gradient centrifugation method will be described.

[Density Gradient Centrifugation Method]

The density gradient centrifugation method is a method of separating particles by using a density difference between components in blood. With the density gradient centrifugation method, it is possible to collect target components (nucleated erythrocytes in the present embodiment) by using a method of not using a separation medium, a method of using one kind of separation medium so as to separate particles into particles on and under the separation medium, a method of using two kinds of separation media so as to separate particles by causing the density range of target components to be sandwiched between the separation media, and the like. By collecting a fraction containing the target components, it is possible to concentrate the nucleated erythrocytes from the maternal blood.

As the method of not using a separation medium, a centrifuge tube is filled with maternal peripheral blood (may be diluted with a diluent) which is a blood sample, centrifugation is performed, and then target components are collected. In this way, the nucleated erythrocytes can be concentrated.

As the method of using one kind of separation medium, a separation medium is injected into the bottom portion of a centrifuge tube, maternal peripheral blood (may be diluted with a diluent) which is a blood sample is laminated on the separation medium, centrifugation is then performed, and the upper portion (may include a portion of the separation medium) of the separation medium having undergone the centrifugation is collected. In this way, the nucleated erythrocytes can be concentrated.

As the method of using two kinds of separation media, a first separation medium is injected into the bottom portion of a centrifuge tube, a second separation medium is laminated on the first separation medium, maternal peripheral blood (may be diluted with a diluent) which is a blood sample is laminated on the second separation medium, centrifugation is then performed, and a layer (may include a portion of the first separation medium and the second separation medium or include a portion of either of the media) between the first separation medium and the second separation medium having undergone centrifugation is collected. In this way, the nucleated erythrocytes can be concentrated. In a case where the centrifuge tube, in which the first separation medium is laminated, is cooled before laminating the second separation medium thereon, it is possible to inhibit mixing that occurs in the boundary region between the first and second separation media.

WO2012/023298A describes the density of maternal blood containing fetal nucleated erythrocytes. According to the description, the density of the fetal nucleated erythrocytes is assumed to be about 1.065 to 1.095 g/mL, the density of the maternal erythrocytes is assumed to be about 1.070 to 1.120 g/mL, the density of the material eosinophils is assumed to be about 1.090 to 1.110 g/mL, the density of the maternal neutrophills is assumed to be about 1.075 to 1.100 g/mL, the density of the maternal basophils is assumed to be about 1.070 to 1.080 g/mL, the density of the maternal lymphocytes is assumed to be about 1.060 to 1.080 g/mL, and the density of the material monocytes is assumed to be about 1.060 to 1.070 g/mL.

The density of the separation medium to be laminated is set such that the fetal nucleated erythrocytes having a density of about 1.065 to 1.095 g/mL is separated from other blood cell components in the mother. For example, in the method of using two kinds of separation media, the central density of the fetal nucleated erythrocytes is about 1.080 g/mL. Therefore, by creating two separation media of different densities between which the cells are sandwiched, and stacking the media and the cells in a state where they are adjacent to each other, desired fetal nucleated erythrocytes can be gathered in the interface therebetween. It is preferable that the density of the first separation medium is set to be equal to or higher than 1.08 g/mL and equal to or lower than 1.10 g/mL, and the density of the second separation medium is set to be equal to or higher than 1.06 g/mL and equal to or lower than 1.08 g/mL. It is more preferable that the density of the first separation medium is set to be equal to or higher than 1.08 g/mL and equal to or lower than 1.09 g/mL, and the density of the second separation medium is more preferably equal to or higher than 1.065 g/mL and equal to or lower than 1.08 g/mL. Specifically, for example, by setting the density of the first separation medium to be 1.085 g/mL and the density of the second separation medium to be 1.075 g/mL, plasma components, eosinophils, and monocytes can be separated from a desired fraction to be collected. Furthermore, some of the erythrocytes, neutrophils, and lymphocytes can also be separated. In the present embodiment, the first separation medium and the second separation medium may be of the same type or different types as long as the effects of the present invention can be realized. However, in a preferred aspect, the media of the same type are used.

As the separation medium for density gradient centrifugation used in the concentration step, it is possible to use a separation medium such as Histopaque (registered trademark) which is a solution containing polysucrose and sodium diatrizoate, Percoll (registered trademark) which is a solution containing silica sol having a diameter of 15 to 30 nm coating nondialyzable polyvinylpyrrolidone, or Ficoll (registered trademark)-Paque which is a neutral hydrophilic polymer solution rich in side chains made from sucrose. In the present embodiment, it is preferable to use Histoqaque and Percoll.

The separation medium for density gradient centrifugation can be prepared at a desired density by being mixed with a diluent or a separation medium having a different density (specific gravity). For example, with Histopaque (registered trademark), a first separation medium and a second separation medium can be prepared at a desired density by using a medium having a density of 1.077 and a medium having a density of 1.119 that are on the market. Furthermore, the osmotic pressure of these media for density gradient centrifugation can be controlled by the addition of sodium chloride (NaCl) and the like.

### EXPLANATION OF REFERENCES

10: flow cytometer
20: container
30: image capturing apparatus
102: nozzle
104: flow cell
106: light source
108, 110: detector
112, 114: deflection electrode plate
120: controller
202: well
204: side wall
206: identification label
208: tube
210: supporting member
212: hole
302: first light source
304: table
306: lens
308: excitation filter
310: dichroic mirror
312: fluorescent filter
314: second light source
316: imaging device
C: cell
L: sheath liquid
S: sample solution
W1, W2, W3, W4, W5, W6, W7, W8, W9, W10, W11: region

What is claimed is:

1. A gene analysis method comprising:
a staining step of staining cells;
a sorting step of obtaining first information derived from cells in a sample solution by using a flow cytometry method, analyzing the first information according to predetermined extraction conditions, and sorting target cells into a container having arrays of a plurality of wells based on the analyzed results;
an amplification step of amplifying DNA of the cells sorted into the container;
an analysis step of performing gene analysis on the amplified DNA; and
a condition determination step of redetermining the extraction conditions based on at least a piece of information between second information obtained in the amplification step and third information obtained in the analysis step.

2. The gene analysis method according to claim 1,
wherein the staining of cells is immunostaining by an antigen-antibody reaction.

3. The gene analysis method according to claim 2,
wherein the first information is at least a piece of information among fluorescence emission, forward-scattered light, and side-scattered light resulting from the immunostaining.

4. The gene analysis method according to claim 1, further comprising:
an imaging step of imaging the cells sorted into the container between the sorting step and the amplification step,
wherein the extraction conditions are redetermined based on fourth information obtained in the imaging step.

5. The gene analysis method according to claim 2, further comprising:
an imaging step of imaging the cells sorted into the container between the sorting step and the amplification step,
wherein the extraction conditions are redetermined based on fourth information obtained in the imaging step.

6. The gene analysis method according to claim 3, further comprising:
an imaging step of imaging the cells sorted into the container between the sorting step and the amplification step,
wherein the extraction conditions are redetermined based on fourth information obtained in the imaging step.

7. The gene analysis method according to claim 4,
wherein the fourth information includes at least one of the fluorescence intensity, shape, color, or size of the cells.

8. The gene analysis method according to claim 1,
wherein the second information includes whether or not the DNA has been amplified, and
the third information includes whether or not the target cells exist.

9. The gene analysis method according to claim 2,
wherein the second information includes whether or not the DNA has been amplified, and
the third information includes whether or not the target cells exist.

10. The gene analysis method according to claim 3,
wherein the second information includes whether or not the DNA has been amplified, and
the third information includes whether or not the target cells exist.

11. The gene analysis method according to claim 1,
wherein the amplification step includes a polymerase chain reaction.

12. The gene analysis method according to claim 2,
wherein the amplification step includes a polymerase chain reaction.

13. The gene analysis method according to claim 3,
wherein the amplification step includes a polymerase chain reaction.

14. The gene analysis method according to claim 1,
wherein the gene analysis is selected from the group consisting of a DNA microarray method, a digital PCR method, a real-time PCR method, a sequencer method, and a combination of these.

15. The gene analysis method according to claim 2,
wherein the gene analysis is selected from the group consisting of a DNA microarray method, a digital PCR method, a real-time PCR method, a sequencer method, and a combination of these.

16. The gene analysis method according to claim 3,
wherein the gene analysis is selected from the group consisting of a DNA microarray method, a digital PCR method, a real-time PCR method, a sequencer method, and a combination of these.

17. The gene analysis method according to claim 1, further comprising:
a concentration step of increasing a concentration of the cells in a solvent before the staining step.

18. The gene analysis method according to claim 2, further comprising:
a concentration step of increasing a concentration of the cells in a solvent before the staining step.

19. The gene analysis method according to claim 3, further comprising:
a concentration step of increasing a concentration of the cells in a solvent before the staining step.

20. A gene analysis method comprising:
a step of staining cells;
a sorting step of obtaining first information derived from cells in a sample solution by a flow cytometry method, analyzing the first information based on predetermined extraction conditions, and sorting target cells into a container having arrays of a plurality of wells based on the analyzed results;
an imaging step of imaging the cells sorted into the container;
an amplification step of amplifying DNA of the cells sorted into the container;
an analysis step of performing gene analysis on the amplified DNA; and
a condition determination step of redetermining the extraction conditions based on at least a piece of information among second information obtained in the amplification step, third information obtained in the analysis step, and fourth information obtained in the imaging step.
